(19) United States
(12) Patent Application Publication
(10) Pub. No.: US 2013/0203695 A1
(43) Pub. Date: Aug. 8, 2013

(54) HYPERSULFATED DISACCHARIDES TO TREAT ELASTASE RELATED DISORDERS

(71) Applicants: Tahir Ahmed, Coral Gables, FL (US); William Abraham, Miami, FL (US)

(72) Inventors: Tahir Ahmed, Coral Gables, FL (US); William Abraham, Miami, FL (US)

(73) Assignee: OPKO Health, Inc., Miami, FL (US)

(21) Appl. No.: 13/880,875

(22) PCT Filed: Oct. 27, 2011
(86) PCT No.: PCT/US11/58085

§ 371 Date: Apr. 22, 2013

Related U.S. Application Data
(60) Provisional application No. 61/408,002, filed on Oct. 29, 2010.

(51) Int. Cl. C13K 13/00; A61K 45/06; A61K 31/7016

(52) U.S. Cl. CPC C13K 13/00; A61K 31/7016; A61K 45/06
USPC 514/53; 536/123.13

(57) ABSTRACT

Hypersulfated disaccharides of formula I and other hypersulfated disaccharides disclosed herein are used to treat diseases or conditions associated with human neutrophil elastase imbalances. The disaccharides and/or intermediates useful to prepare such compounds are prepared from heparin. The diseases and conditions which are treated with a compound of formula I include chronic obstructive pulmonary disorder (COPD) and cystic fibrosis (CF). The formulations are delivered to the lungs in an aerosol formulation or dry powder means or via nebulization. Oral forms are also suitable.

### FIELD OF THE INVENTION

The present invention relates to the use of a hypersulfated disaccharide compound of formula I as further described below and other hypersulfated disaccharides as disclosed herein in the treatment of diseases or conditions associated with leukocyte elastase. In particular, the present invention relates to formulations of a compound of formula I to improve lung function (tracheal mucous velocity) and/or to treat/mitigate diseases or conditions such as chronic obstructive pulmonary disease (COPD) and/or cystic fibrosis (CF). COPD has been described as a “quiet killer” because of its slow progression and the fact that it is often untreated during the early course of the disease. Emphysema and chronic bronchitis are sub-types of COPD. In emphysema, the walls of the alveoli are structurally damaged which ultimately reduces the surface area for gas exchange and lung capacity. Chronic bronchitis is characterized by excessive mucous production and airflow limitations develop with disease progression. Patients with COPD have significant airflow limitations and eventually lose the ability to adequately oxygenate the blood. COPD is a leading cause of death worldwide and the rate of COPD-related deaths is rising. New England J. of Med. Sep. 16, 2010. Progressive loss of lung function, a hallmark of COPD, is not prevented by currently available therapy. There is thus a severe need for drugs or effective treatments for this disease.

Elastases are typically released from leukocytes such as macrophages and neutrophils and contribute to the significant structural damage caused in COPD. Human neutrophil elastase (HNE) is known as a very potent protease that can degrade the macromolecular components of connective tissue such as elastin, induces mucus hypersecretion and causes or is associated with diseases such as COPD, CF and other inflammatory disorders such as rheumatoid arthritis. Elastase is also known to bind to adhesion molecules such as Mac-1 which regulates or participates in neutrophil adhesion and transmigration. In addition, elastase can cleave intercellular adhesion molecule 1 (ICAM-1) which is a ligand for Mac-1. Thus, increased elastase load in the lungs of COPD patients, which is not altered by current available therapy including steroids, creates the need for elastase inhibitors and/or other pharmaceutical therapy that reduces the impact of elevated levels of elastase in COPD, CF and other such diseases. For COPD, the disease is currently treated with inhaled anticholinergic bronchodilating agents (ipratropium bromide, tiotropium) or inhaled beta agonists (albuterol, salmeterol or formoterol) or the combination of such agents with steroids (Advair®, Symbicort®) or methylxanthines (theophylline). For CF, the current therapy includes DNASE, inhaled antibiotics (e.g. tobramycin), anti-inflammatory agents (e.g. high dose ibuprofen) along with the above treatment(s) for COPD. There is a significant need for new therapies, which would effectively treat and/or mitigate these diseases.

### BACKGROUND OF THE INVENTION

There are a multitude of patents and scientific publications that disclose or relate to attempts to find inhibitors of HNE that would effectively treat chronic or genetic lung disorders that typically have severe symptoms associated with the powerful and damaging effects of elastase. Among the class of drugs known as elastase inhibitors, heparin or derivatives thereof have been the focus of significant interest. Heparin is extremely potent against HNE, both in vitro and in vivo. This potency and relative activity is apparently due to the specific chemical properties of heparin's molecular structure. These properties include mass, chain length, degree of sulfation, charge density, specific sulfation and iduronic acid content. Heparin is also known to affect leukocyte interactions with vascular endothelium and it also affects the release of elastase in addition to being an inhibitor of elastase. It is also known that heparin has anticoagulation activity so the present inventors along with several other scientists have discovered heparin analogs or derivatives thereof including short oligosaccharides derived from heparin that have anti-inflammatory activity without having anticoagulant properties.

Previous findings have suggested that for elastase inhibitory activity of heparin fragments, a chain length of at least 12-14 saccharides is required. Other papers have suggested that a minimum molecular weight (M) of 2000-3000 of a heparin moiety is necessary to inhibit elastase. Supersulfated heparins (6.3 kD) have been investigated with respect to their elastase inhibition activity. In studies related to evaluating the effects of heparin on adhesion activity, it was found that heparin fractions of 4-14 saccharides had no effect on the adhesion of neutrophils to endothelial cells that had been stimulated with IL-1beta. Inhibition of elastase release by heparin oligosaccharides follows a similar pattern. Heparin oligosaccharides having 4-, 6- or 8-saccharides only had nominal effect at high concentrations and inhibitory activity was lost as molecular weight decreased. The present inventors have surprisingly discovered that a short length, low molecular weight polysulfated disaccharide of formula I treats or mitigates the effects of human neutrophil elastase and is thus useful as a drug to treat conditions or diseases associated with elevated elastase activity or an imbalance of elastase/anti-elastase activities.

U.S. Pat. No. 7,056,898 (the '898 patent) discloses and claims certain hypersulfated disaccharides and methods of using same to treat certain inflammatory disorders. The '898 patent specifically describes the use of the claimed compounds to treat pulmonary inflammations including asthma and asthma-related pathologies, such as allergic reactions or an inflammatory disease or condition. The compounds disclosed therein are described as being capable of preventing, reversing and/or alleviating the symptoms of asthma and asthma-related pathologies, particularly the late phase response in asthma patients following antigen stimulation.

U.S. Provisional 61/266,361 discloses that certain formulations comprising the hypersulfated disaccharides recited herein and a delivery agent selected from the group consisting of a pharmaceutically acceptable natural or synthetic polymer as well as other vehicles that heretofore have been utilized to improve delivery of large compounds (e.g., those compounds having molecular weights of greater than 4,500 daltons as average molecular weight) have enhanced absorption/bioavailability/efficacy relative to the same compounds delivered orally without the claimed additives.

### SUMMARY OF THE INVENTION

The present invention relates to pharmaceutical formulations comprising a compound of formula I and pharmaceutically acceptable salts thereof and a vehicle suitable for inhalation,

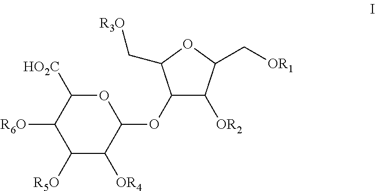

wherein R₁, R₂, R₃, R₄, R₅ and R₆ are independently selected from the group consisting of H, SO₃H or PO₃H and provided that at least two of R₁-R₆ is selected from SO₃H or PO₃H. The present invention further relates to formulations having compounds of formula I wherein at least three of R₁-R₆ are selected from SO₃H or PO₃H. The present invention further relates to formulations having compounds of formula I wherein at least four of R₁-R₆ are selected from SO₃H or PO₃H. The present invention further relates to formulations having compounds of formula I wherein at least five of R₁-R₆ are selected from SO₃H or PO₃H. The present invention preferably relates to a compound of formula I and pharmaceutically acceptable salts thereof wherein R₁-R₆ are selected from SO₃H. The present invention also relates to formulations having a compound of formula I wherein R₁-R₆ are independently selected from SO₃H or PO₃H. The invention further includes pro-drugs, derivatives, active metabolites, partially ionized and fully ionized derivatives of the compounds of formula I and stereoisomers thereof. The monomers which make up the disaccharides of the invention may be D or L isomers and the hydroxyl moieties or sulfated or phosphated versions thereof around the carbocyclic ring (or intermediates thereof) may have the alpha or beta designation at any particular stereocenter. The linking oxygen atom between the monosaccharide moieties may also be alpha or beta. The molecular weight of the compounds of the invention is typically less than 1,000 daltons. The present invention also relates to the use of polysulfated disaccharides having two six-membered rings in the treatment of elastase related disorders.

The most preferred embodiment relates to an aerosol/nebulizable formulation containing a compound of formula I and pharmaceutically acceptable salts thereof wherein R₁-R₆ are selected from SO₃H. The present invention also relates to oral formulations of a compound of formula I with the variables as defined above for the treatment of elastase related disorders.

The present invention also encompasses a method of treating an elastase-associated condition in an organism in need of treatment thereof comprising administering a pharmaceutically effective amount of a compound comprising a compound of formula I

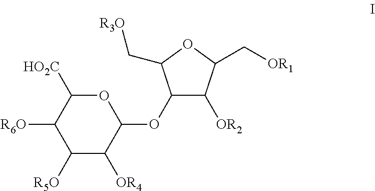

and pharmaceutically acceptable salts thereof wherein R₁-R₆ are independently selected from SO₃H, PO₃H or H and provided that at least two of R₁-R₆ is SO₃H or PO₃ .

### BRIEF DESCRIPTION OF THE DRAWINGS

The invention will be described in the following drawings.

FIG. 1 illustrates the effects of inhaled hypersulfated disaccharide on the HNE-induced reduction in Tracheal Mucus Velocity (TMV).

FIG. 2 shows the effects of equivalent doses of disaccharide sodium on the HNE-induced effects.

FIG. 3 illustrates that hypersulfated disaccharide can also reverse the effects of HNE.

FIG. 4 illustrates the positive effects of oral hypersulfated disaccharide in a Carbopol formulation on HNE-induced Reduction in TMV.

### DETAILED DESCRIPTION

The present invention relates to pharmaceutical formulations suitable for delivery to the lungs of a patient in need of such treatment and uses thereof wherein the formulation comprises a compound of formula I and pharmaceutically acceptable salts thereof

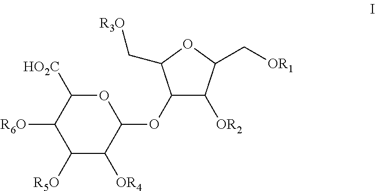

wherein R₁, R₂, R₃, R₄, R₅ and R₆ are independently selected from the group consisting of H, SO₃H or PO₃H and provided that at least two of R₁-R₆ is selected from SO₃H or PO₃H.

The present invention also relates to a pharmaceutical formulation suitable for delivery to the lungs of a patient in need of such treatment comprising (i) a compound of formula I and pharmaceutically acceptable salts thereof

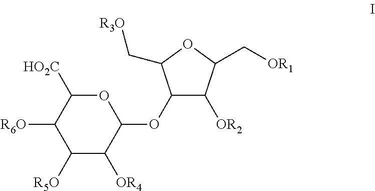

wherein R₁, R₄ and R₅ are independently selected from H, SO₃H or PO₃H and R₂, R₃ and R₆ are independently selected from SO₃H or PO₃H.

The present invention also relates to a pharmaceutical formulation suitable for delivery to the lungs of a patient in need of such treatment comprising (i) a compound of formula I and pharmaceutically acceptable salts thereof

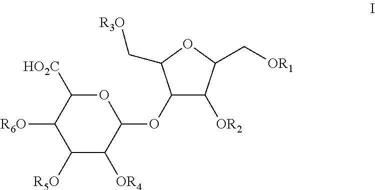

wherein R₂ and R₆ are independently selected from H, SO₃H or PO₃H and R₁, R₃, R₄ and R₅ are independently selected from SO₃H or PO₃H.

The invention relates to a pharmaceutical formulation suitable for delivery to the lungs of a patient in need of such treatment comprising (i) a compound of formula I and pharmaceutically acceptable salts thereof

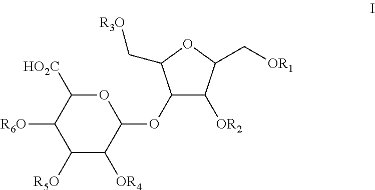

wherein R₁, R₂ and R₆ are independently selected from H, SO₃H or PO₃H and R₃, R₄ and R₅ are independently selected from SO₃H or PO₃H.

In another embodiment, the present invention relates to a pharmaceutical formulation suitable for delivery to the lungs of a patient in need of such treatment comprising (i) a compound of formula II

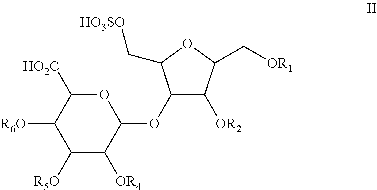

and pharmaceutically acceptable salts thereof wherein R₁, R₂, R₄, R₅ and R₆ are independently selected from the group consisting of SO₃H or PO₃H.

In a preferred embodiment, the invention relates to a pharmaceutical formulation suitable for delivery to the lungs of a patient in need of such treatment comprising (i) a compound of formula II and pharmaceutically acceptable salts thereof

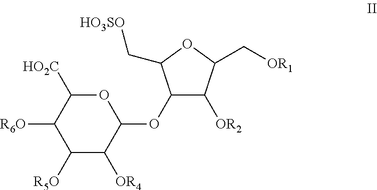

wherein R₁ and R₄ are SO₃H and R₂, R₅ and R₆ are independently selected from H, SO₃H or PO₃H.

In an additional preferred embodiment, the invention relates to a pharmaceutical formulation suitable for delivery to the lungs of a patient in need of such treatment comprising (i) a compound of formula II and pharmaceutically acceptable salts thereof

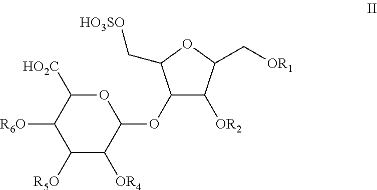

wherein R₁ is SO₃H, R₂ is H and R₄, R₅ and R₆ are independently selected from SO₃H or PO₃H.

The present invention also relates to liquid or solid dosage forms suitable for delivery to the lungs of a patient in need of such treatment comprising a compound of formula I or II and their pharmaceutically acceptable salts with R₁-R₆ as defined above.

The present invention also encompasses a method of treating or alleviating a condition associated with elastase or in imbalance of elastase/anti-elastase comprising administration of (i) a pharmaceutically effective amount of a formulation comprising a compound of formula I

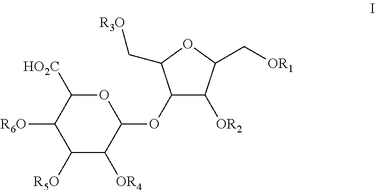

and pharmaceutically acceptable salts thereof wherein R₁-R₆ are independently selected from SO₃H, PO₃H or H and provided that at least two of R₁-R₆ is SO₃H or PO₃H.

The present invention preferably relates to a nebulizable, dry-powder or aerosol pharmaceutical formulation comprising a compound of formula I wherein R₁, R₂, R₃, R₄, R₅ and R₆ are selected from the variables shown in Table 1 as compounds 1-14.

TABLE 1

| | |
|---|---|
| | I |
| 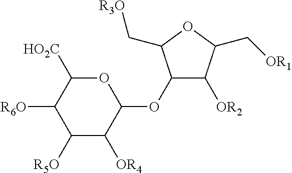 | |

| Compound | R₁ | R₂ | R₃ | R₄ | R₅ | R₆ |
|---|---|---|---|---|---|---|
| 1 | —SO₃H | H | —SO₃H | —SO₃H | H | H |
| 2 | H | —SO₃H | —SO₃H | —SO₃H | H | H |
| 3 | H | H | —SO₃H | —SO₃H | H | —SO₃H |
| 4 | —SO₃H | H | —SO₃H | —SO₃H | —SO₃H | H |
| 5 | —SO₃H | H | —SO₃H | —SO₃H | H | —SO₃H |
| 6 | H | H | —SO₃H | —SO₃H | —SO₃H | —SO₃H |
| 7 | —SO₃H | H | —SO₃H | —SO₃H | —SO₃H | —SO₃H |
| 8 | —SO₃H | —SO₃H | —SO₃H | —SO₃H | H | H |
| 9 | H | —SO₃H | —SO₃H | —SO₃H | —SO₃H | H |
| 10 | H | —SO₃H | —SO₃H | —SO₃H | H | —SO₃H |
| 11 | —SO₃H | —SO₃H | —SO₃H | —SO₃H | —SO₃H | H |
| 12 | —SO₃H | —SO₃H | —SO₃H | —SO₃H | H | —SO₃H |
| 13 | H | —SO₃H | —SO₃H | —SO₃H | —SO₃H | —SO₃H |
| 14 | —SO₃H | —SO₃H | —SO₃H | —SO₃H | —SO₃H | —SO₃H |

In a preferred embodiment, the compounds in the formulation are selected from a metal salt of a compound of formula I shown above in Table 1 wherein the carboxylic acid group is ionized and each sulfate group around the disaccharide is ionized to form a metal salt wherein the metals are selected from, for example, sodium. In addition, other salts including amine salts may form at the carboxylate or sulfate positions. The most preferred compound is the fully ionized form as the sodium salt of compound 14 (compound 14a).

The compounds of the invention may be obtained as described herein in the examples from, for example, heparin. Although the specific process used utilized porcine heparin, heparin from any mammal may be used to produce the compounds of the invention. In addition, the compounds may be derived synthetically. Various other polysaccharides may also be utilized as source materials for the recited disaccharides including, but not limited to, heparan sulfate, dermatan sulfate, chondroitin sulfate, pentosan polysulfate and other glycosaminoglycans and mucopolysaccharides.

The compounds can generally be prepared by a process which comprises (1) dissolving heparin sodium in water and adjusting the pH to be slightly acidic (about pH 6) and (2) treating this solution with sodium nitrite (NaNO₂) in an aqueous solution to form nitrous acid to depolymerize the heparin (and deaminate, for example, IdoA(2S)GlcNS(6S) to form IdoA(2S)-aMan) and (3) basifying the depolymerized heparin solution to a pH of about 7 and (4) diluting the depolymerized heparin solution and (5) filtering said solution to collect and enrich for heparin oligosaccharides of less than 3 kDa (3000 daltons) and (6) basifying the filtered solution containing less than 3 kDA depolymerized heparin and (7) treating this basified solution with sodium borohydride (NaBH₄) to reduce the aldehyde carbonyl, formed after the acidification of and depolymerization of heparin, to the alcohol; (8) treating the reduced product with concentrated acid and then adjusting the pH to about 7 and (9) further fractionating the obtained reduced oligomers using size exclusion chromatography to obtain disaccharide ammonium salts which were further treated with cation exchange resins to form the sodium salts which were further fractionated to obtain, as a major component, a compound of formula I as the sodium salt form wherein R₁ is H, R₂ is H, R₃ is SO₃⁻, R₄ is SO₃⁻, R₅ is H and R₆ is H and the carboxy group (CO₂H) is CO₂⁻Na⁺ and, as a minor component, a compound of formula I wherein R₁ is H, R₂ is H, R₃ is SO₃⁻, R₄ is SO₃⁻, R₅ is SO₃⁻ and R₆ is H and the carboxy group (CO₂H) is CO₂⁻Na⁺ and (10) treating the resulting disaccharides with a sulfate source (e.g. (CH₃)₃NSO₃) under suitable conditions to form the hypersulfated disaccharides utilized in the formulations of the invention.

Without being limited herein, it is understood that heparin and other carbohydrates or complex carbohydrates are chiral molecules with hydroxyl groups as well as sulfate groups or carboxylic acid groups present on the ring with set or absolute stereochemistry. The most common disaccharide unit in heparin is, for example, IdoA(2S)-GlcNS(6S) which is a 2-O-sulfated iduronic acid and 6-O-sulfated glucosamine.

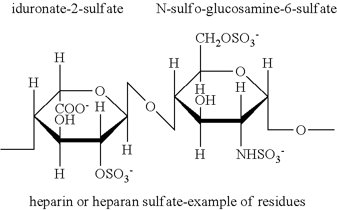

It is generally understood that the source of the polysaccharide which generates the oligosaccharides and disaccharides utilized in the formulations of the invention will determine, for the most part, the absolute stereochemistry of the chiral centers around the carbohydrate rings. Additional sulfate groups are added by chemical means by the process described generally above or by any known means to afford the most active moieties (hypersulfated disaccharides and salts thereof) which are further purified to form pharmaceutical grade disaccharides which are further formulated with an excipient to form a formulation suitable for delivery to the lungs of a patient in need of treatment thereof. The molecule shown above as a polysulfated derivative will be active in the treatment of elastase-related disorders. Such polysulfated derivative would have more than the three sulfate groups shown above.

Nuclear magnetic resonance imaging and/or other known structure identification methods may be used to determine the chemical structures of the molecules obtained from depolymerizing heparin (derived from any known source thereof) or other selected polysaccharide. In the event the compounds are made synthetically or semi-synthetically, the skilled artisan can use standard organic chemistry techniques to protect the desired hydroxyl moiety with a protecting group known to those of ordinary skill in the art.

A compound of formula I as described above (or mixtures thereof) is then formulated with aerosol excipients or nebulizable excipients to form the formulations of the invention. The excipient is selected from the group consisting of any known or discovered inhalant, propellant and/or other additives that are suitable to deliver to the lungs of a patient. Such formulations and/or active ingredient described herein may also be delivered with or combined with or used in combination with known treatments for COPD, a sub-disease thereof, CF or other elastase related conditions. For COPD, the disease is currently treated with inhaled anticholinergic bronchodilating agents (ipratropium bromide, tiotropium) or inhaled beta agonists (albuterol, salmeterol or formoterol) or the combination of such agents with steroids (Advair, Symbicort) or methylxanthines (theophylline). For CF, the current therapy includes DNASE, inhaled antibiotics (e.g. tobramycin), anti-inflammatory agents (e.g. high dose ibuprofen) along with the above treatments) for COPD. Depending upon the patient and the physician's prescription thereof, the combination of the present invention and any one of the above-treatments for the named diseases may be used to treat a patient. Steroids are typically not effective for COPD patients so there is a tremendous need for therapy such as the claimed polysulfated disaccharide formulations.

The formulations of the invention can be delivered to the patient or other organism by any suitable known means. The percentages of the additive and type of additive added to the formulation relative to the active ingredient and other excipients will be based upon the type of formulation desired. For example, in an aerosol formulation, suitable propellants as well as aqueous solutions may be employed to deliver the drug in a suitable delivery device such as an inhaler.

The compositions of the invention further comprise pharmaceutically acceptable excipients suitable for aerosol delivery means or nebulizable means.

The compounds of formula I and II form, as stated above, pharmaceutically acceptable salts. The metal salts include for example salts having Na, K, Ca, Ng or Ba or Al, Zn, Cu, Zr, Ti, Bi, Mn or Os or salts formed by reacting the compounds of formula I or II with an organic base such as an amino acid or with any amine. The preferred salt is a sodium salt.

Thus, the preferred formulations of the invention includes those compounds shown in Table 1 and which are hypersulfated disaccharides and which further include a delivery agent selected from, for example, an aqueous nebulizable solution. The preferred active ingredient is in the form of a sodium salt wherein sodium replaces the carboxylic hydrogen atom in formula I.

These formulations are useful in treating those conditions associated with an elevated or abnormal level of human neutrophil elastase such as COPD, cystic fibrosis and the like.

Cystic fibrosis is characterized by the production in patients lungs of an abnormally viscous mucus which leads to chronic infection by pathogenic bacteria. The bacterial colonies initiate an influx of inflammatory cells which further cause an elevation in inflammatory cytokines (IL-6 and IL-8). This results in a recurring cycle of infection and inflammation in CF patients and leads to morbidity and mortality. In CF patients, a drug which is mucolytic and permits clearing of the mucus from the lungs and which also has anti-inflammatory and anti-elastase activity would help mitigate or treat this disease. While heparin has been used to treat CF patients in a small clinical trial (six patients) with some success with respect to thinner sputum and other clinical parameters, there is no approved use of this medicine to treat CF or other elastase related conditions such as emphysema or COPD.

As used herein, the term “treating or alleviating the symptoms” means reducing, preventing and/or reversing the symptoms of the individual to which a formulation of the invention has been administered as compared to the symptoms of the individual or an individual which is untreated.

The formulations of the invention have been found to be effective in animal studies which are predictive of utility in humans as well as other animals. The particular animal studies described herein demonstrate that the formulations are useful in improving or stimulating whole lung mucociliary clearance (MCC). Tracheal mucus velocity (TMV) is measured as a marker of MCC in sheep. The TMV model is used under various relative conditions as a measurement to mimic responses observed in, for example, impaired individuals and impaired individuals being treated with drug. Neutrophil elastase is used in the animal models (sheep) as an agent that induces mucociliary dysfunction and as an agent that depresses MCC for up to 8 hours in sheep. The compound of formula I was then used as a medicament to increase TMV and restore MCC in the elastase treated animals.

Sheep used in the studies were treated with humane care. The sheep were conscious throughout the studies and instrumentation was performed after treating the animals with a local anesthetic. To study the effects of a compound of formula I, the sheep after being topically treated with anesthetic (2% lidocaine in nasal passages) were nasally intubated with an endotracheal tube (7.5 cm in diameter). The cuff of the tube was placed just below the vocal cords to permit maximal exposure of the tracheal surface area. The cuff was deflated throughout the study period except during the period of drug delivery in order to minimize impairment of TMV by the tube. The inspired air was warmed and humidified.

TMV was measured in vivo by the methods generally described in the publication Chest, Vol 128/5/November 2005, pp 3742-3749. TMV was measured in vivo by a roentgenographic technique using five to 10 radiopague Teflon/bismuth trioxide disks, 1 mm in diameter, 0.8-mm thick, and 1.8 mg in weight. The disks were insufflated into the trachea via a modified suction catheter connected to a source of continuous compressed air (3-4 L/min). The catheter remains in the endotracheal tube and no contact with the tracheal surface is made. The cephalid-axial velocities of the individual disks are recorded on videotape from an image intensifier unit. Individual disk velocities are calculated by measuring the distance traveled by each disk during a 1-min observation period. For each run, the mean value of all individual disk velocities is then calculated. The sheep used in the studies wore collars containing radiopaque reference markers of known length as a standard to correct for magnification errors inherent in the fluroroscopy unit.

HNE was obtained from Elastin Product Company (Owensville, Mo.). A stock solution was prepared according to the specifications of the manufacturer. Sheep were administered the stated amount of HNE using a Raindrop Nebuilizer (Nellcor Puritan-Bennett, Carlsbad, Calif.) aerosol delivery system which produces a droplet with a MMAD of approximately 1.1 micrometers. The nebulizer was connected to a dosimeter consisting of a solenoid valve and a source of compressed air at 20 pounds per square inch (psi). The output of the nebulizer was connected to a T-piece, with one end attached to a Harvard respirator (Harvard Apparatus Inc., Holliston Mass.). The respirator was set at an inspiratory/expiratory ratio of 1:1 and a rate of twenty breaths per min. The solenoid valve was activated for one second at the beginning of the respiratory cycle of the respirator. A Tidal volume of 500 ml was used to deliver the agents. In the present invention, any suitable means to deliver a compound of formula Ito the lungs of a patient may be used. Aerosol delivery means, nebulizable delivery means and propellant and/or inhalant device means are known in the art and may be utilized herein. The compounds utilized herein are preferably used as dry powders that are then prepared as a solution on the day of delivery to the patient using a sterilized container and deionized water or other suitable solvent/delivery system. In some devices, dry powders of a compound of formula I may be utilized to deliver medicine to the patient without the need for solublizers or solutions.

The formulations of the invention may also be administered in combination with other suitable medications or active ingredients and depending upon the particular disease or condition being treated. The present invention relates to a method of treatment of COPD comprising administering to an organism in need thereof a therapeutically effective amount of a compound of formula I or II with R₁-R₆ as defined herein (i.e., with at least two sulfate groups). The additional active ingredients that may be administered in the form of combination therapy or in the form of a single dosage unit having at least two active ingredients wherein the first active is a compound of formula I or II with R₁-R₆ as defined herein and a second active selected from any drug or medicament which is used as front line therapy to treat any condition that is secondary to CF, COPD or any elastase related condition or disorder. Such medicaments include anti-inflammatory agents, leukotriene antagonists or modifiers, anticholinergic drugs, mast cell stabilizers, corticosteroids, immunomodulators, beta-adrenergic agonists (short acting and long acting), methyl xanthines, and other general classes or specific drugs used to treat such disorders including, but not limited to, montelukast sodium; albuterol; levoalbuterol; salmeterol; formoterol, fluticasone propionate; budesonide; ceterizine; loratadine; desloratadine; theophylline, ipratropium, cromolyn, nedocromil, beclomethasone, flunisolide, mometasone, triaminoclone, prednisoline, prednisone, zafirlukast, zileuton or omalziunab.

The following examples are intended to further illustrate certain embodiments of the invention and are non-limiting.

### EXAMPLE 1

### Preparation of Hypersulfated Disaccharides

The compounds utilized in the formulation of the invention were prepared by initially depolymerizing heparin sodium. The starting material for preparation of the active drug substance is, for example, porcine intestinal mucosal hepartin (polydisperse sulfated copolymer of 1 to 4 linked glucosamine and uronic acid residues). The active drug substance (ADS), a hypersulfated disaccharide, as described herein was shown to have anti-allergic activity in the sheep model. The production of the ADS was generally as follows:

1) Controlled nitrous acid depolymerization of porcine heparin;

2) Reduction of the end aldehyde group with NaBH₄ to an alcohol;

3) Size exclusion chromatography (SEC) to produce the ammonium salt of the separated disaccharide;

4) Reaction of the disaccharide ammonium salt with sulfur trioxide pyridine complex to yield the supersulfated disaccharide;

5) SEC followed by cation exchange to the sodium salt afforded the final product

The preferred product produced in this way was the hypersulfated disaccharide having six sulfate groups in the sodium salt form as shown below (compound 14a)

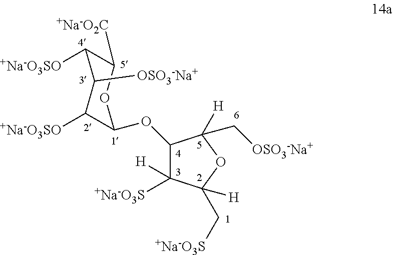

Compound 14a has a solubility of >0.5 g/mL. The following procedure describes one of many possible ways to make the compounds described herein. At room temperature, 250 g of commercially available porcine heparin-Na (obtained from commercially available sources including, for example, SPL of Waunakee, Wis.) were added to a beaker containing three liters of water and stirred to a slurry, at which point two additional liters of water were added to completely dissolve the heparin salt.

The pH in the heparin solution was then adjusted to about pH 6 (5.98). To this solution was added 17.25 g of NaNO₂ (0.25 mmol, J.T. Baker, ACS grade) to accomplish the controlled nitrous acid depolymerization of the heparin. Stirring was continued for 10 minutes while approximately 35.1 ml of 37% HCl was slowly added at a temperature of about 23° C. to bring the pH to about 3 (3.00). The temperature and pH of the solution was monitored over a two hour period (120 minutes) while the temperature went down to 20° C. and the pH went down to pH 2.16. The solution was then quenched by slowly adding approximately 23 ml of 50% NaOH to adjust the pH to 6.75 to afford the depolymerized heparin solution.

The depolymerized heparin solution obtained above was diluted to a final volume of 8 liters with dtH₂O and filtered (Millipore (Bedford, Mass.), Pellicon 2, 3 k PLBC-C having an area of 0.5 m2 (Cassett: Cat # P2 PLBCC 05), (molecular weight cut off of 3 kDa) to collect and enrich for heparin oligosaccharides of less than 3 kDa (3000 daltons) in size (i.e., the permeate consisted of those oligosaccharides of less than 3000 daltons). The retentate that was larger than 3000 daltons was subjected to a second depolymerization treatment of nitrous acid using a 20 M solution to farther initiate the degradation of heparin. After ultrafiltration of this twice-treated oligosaccharide preparation using the same type of filter (molecular weight cut off of 3,000 daltons), the resulting permeate (with a molecular weight of less than 3 kDa) was added to the permeate from the first ultrafiltration and then the entire batch was concentrated by reverse osmosis to reduce the final volume to 2.5 liters. This was then freeze dried.

The freeze-dried oligosaccharide preparation (50 g) was dissolved in 1 liter purified water and then cooled in an ice bath to 2-10° C. NaHCO₃ (21 g) was added to the cooled oligosaccharide solution and the preparation stirred until completely dissolved. A 0.5 M solution of sodium borohydride (NaBH₄) in 400 mL of 0.01 M NaOH solution was prepared and slowly added to the cooled oligosaccharide/NaHCO₃ solution over a 60 minute period. The treatment of 0.5 M solution of NaBH₄ was to reduce the aldehyde formed on the five membered ring (which formed after deamination) to the alcohol moiety. The reaction preparation was stirred at 2-10° C. for 3 hours, then quenched with concentrated HCl to pH 4.0. The pH of the solution was then adjusted to 6.75 with NaOH and finally concentrated to a minimal volume by reverse osmosis and later freeze-dried to afford the reduced oligosaccharides. The reduced oligosaccharide preparation of less than 3 kDa in size were later subjected to fractionization by size exclusion chromatography (SEC) using Bio-Rad Biogel P6 resin (elution with 0.2 M NH₄HCO₃) for the fractionization of the oliogmix and to collect disaccharide ammonium salts. The collected fractions were analyzed by carbazole assay, a plot of Abs₅₃₀ versus fraction number afforded a profile of collected fractions. Similar fractions on profile were pooled and later lyophilized to afford the separated fractions as ammonium salts and to remove NH₄HCO₃. Cation exchange using Amberlite IR 120 Plus cation exchange resin (commercially available from Sigma-Aldrich) converted to ammonium salt(s) to the sodium salt form. Two disaccharides were obtained from the fractions and were identified as compounds A (85 wt %) and B (3-5 wt %):

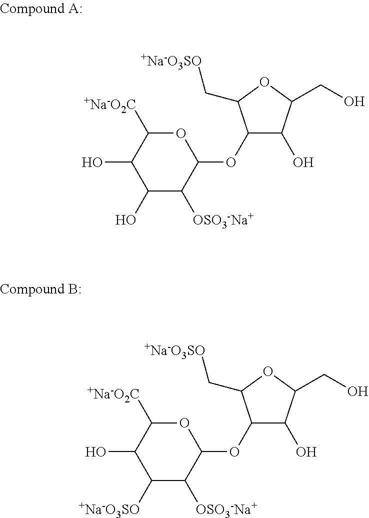

Fractions containing the above compounds A and B were further treated to form the hypersulfated disaccharides. Two non-limiting methods were utilized. In Method 1, a solution of the above fraction containing 2.5 grams disaccharide in 50 mL water was acidified through reaction with Dowex 500WX200 acidic resin commercially available from Sigma-Aldrich according to the manufacturer's instructions. The acidic filtrate was neutralized with tetrabutylammonium hydroxide and the solution was freeze-dried to obtain the tetrabutylammonium (Bu₄N+) salt as a flocculent solid. Anhydrous DMF (50 mL) was then added to a mixture of the disaccharide ammonium salt and (CH₃)₃NSO₃ (5.22 grams) under Argon. The reaction mixture was heated at 50° C. for 48 hours. The solution was then cooled to room temperature. 100 mls of a saturated solution of sodium acetate in ethanol was added and the mixture was stirred for twenty minutes at room temperature, diluted with 2.5 L of water and then filtered against a 500 dalton (i.e., 0.5 kDa) membrane. The retentate (i.e., larger than 0.5 kDa) was freeze-dried; resuspended in 0.2 M NH₄HCO₃ solution, chromatographed on Bio-Rad Biogel P6 resin (Bio-Rad, Hercules, Calif.) according to the manufacturer's instructions and eluted with 0.2 M NH₄HCO₃ to obtain the NH₄⁺ salt of the hypersulfated disaccharide (3.5 grams). A portion of this salt (2.4 grams) was converted to the Na⁺ salt form through reaction with Amberlite IR 120 Plus cation exchange resin (commercially available from Sigma-Aldrich) according to the manufacturer's instructions to afford the sodium salt of compound 14 shown in Table 1 and shown below as compound 14a:

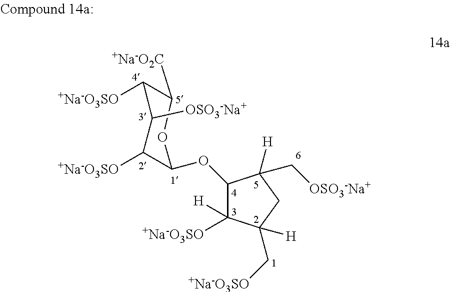

This compound was also prepared according to Method 2. In Method 2, a mixture of 0.5 grams of the fraction containing compounds A and B and 3 grams of (CH₃)₃NSO₃ in 15 mL DMF under Argon was heated at 60° C. for 48 hours. The reaction mixture was then cooled to room temperature, diluted with 20 mL of a 10% aqueous sodium acetate solution, and stirred 20 minutes at room temperature, 100 mL of ethanol was added and the reaction mixture was concentrated under high vacuum to obtain a solid residue. The residue was dissolved in 500 mL of water and filtered against a 500 dalton membrane (washing 3× with H₂O). The sodium salt retentate which contained the hypersulfated 14a product was freeze-dried to an off-white solid.

### EXAMPLE 2

To illustrate the effectiveness of the formulations according to the invention to treat and alleviate elastase related diseases and conditions, including but not limited to the specific diseases and conditions recited herein, Applicant is providing the following examples:

### Animal Preparation

All procedures used in this study were reviewed and approved by the Mount Sinai Medical Center Animal Research Committee, which is responsible for ensuring the humane use of experimental animals.

Adult sheep were restrained in an upright position in specialized body harness in carts. The head of the animals were immobilized, and after local anesthesia of the nasal passage was induced with 2% lidocaine, the animals were nasally intubated with a standard endotracheal (ET) tube (7.5 mm diameter, Mallinckrodt, St. Louis, Mo.). A fiberoptic bronchoscope was used to guide the ET tube and verify its position in the trachea. The cuff of the tube was placed just below the vocal cords to allow for maximal exposure of the tracheal surface area. To minimize possible impairment of TMV caused by endotracheal tube cuff inflation, we deflated the cuff throughout the study except for the period during nebulization of compounds. Additionally, to alleviate the effects of prolonged intubation, we warmed and humidified the inspired air using a Bennett Humidifier (Puritan-Bennett; Lenexa, Kans.). After intubation, the animals were allowed rest for approximately 20 minutes before beginning the study. The animals were awake and alert throughout the entire study.

### TMV Measurements

TMV was measured in vivo by fluoroscopic technique utilizing a Siremobile 2000 fluoroscope (Siemens). Five to seven radiopaque Teflon/bismuth trioxide disks (1 mm in diameter, 0.8-mm in thickness, and 1.8 mg in weight) were insufflated into the mid-portion of the animal's trachea. A catheter connected to a source of continuous compressed air at 3 to 4 L/min, was used to deliver the discs on to surface of the trachea via the endotracheal tube. The catheter remained within the endotracheal tube only during insufflation of the disks and made no contact with the tracheal surface. Once the disks were delivered onto the trachea, the cephalad-axial velocity of each individual disk was recorded on videotape from a portable image intensifier unit in-line with the fluoroscope. The velocities were calculated by measuring the distance traveled by each disk during a 1-min observation period. For each run, the mean value of all individual disk velocities was calculated. A collar containing radiopaque reference markers of known length was secured around the sheep's neck and was used as a standard to correct for magnification effects inherent in the fluoroscopy unit.

### Aerosol Delivery System

All agents were aerosolized using a Raindrop Nebulizer (Nellcor Puritan-Bennett, Carlsbad, Calif.). which produces a droplet with a MMAD of approximately 1.1 micrometers. The nebulizer was connected to a dosimeter system consisting of a solenoid valve and a source of compressed air at 20 pounds per square inch (psi). The output of the nebulizer was connected to a T-piece, with one end attached to a Harvard respirator (Harvard Apparatus Inc., Holliston, Mass.). The respirator was set at an inspiratory/expiratory ratio of 1:1 and a rate of 20 breaths/minute. The solenoid valve was activated for 1 second at the beginning of the inspiratory cycle of the respirator. A tidal volume of 500 ml was used to deliver agents.

### Agents

Human Neutrophil Elastase (HNE) was obtained from Elastin Products Corporation, Inc. (Owenville, Mo.). A stock solution was prepared according to the specifications of the manufacturer. Aliquots of 6.8 micoliters containing 1190 mU of active enzyme were prepared from stock and stored at −80° C. On the day of the experiment, the HNE was dissolved in 3 mL phosphate-buffered saline solution, and the sheep were administered the total amount using the aerosol delivery system described above.

Disaccharide sodium and hypersulfated disaccharide were provided as dry powders. Solutions were prepared fresh on the day of the experiment. A sterilized container was used to weigh the compounds and a total of 3.0 mL of deionized water was added into the container. Once the compounds were completely dissolved, the solution was administered to the animals by aerosol using the system described. All agents were nebulized to dryness (approximately 10-12 minutes).

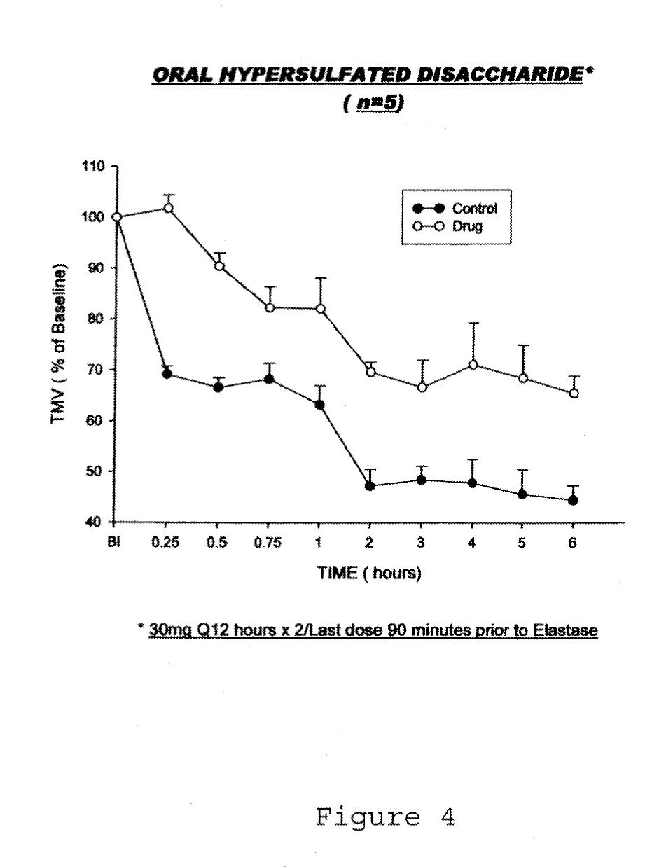

Oral dosage forms in the form of capsules were prepared using a 1:2 ratio of active ingredient to Carbopol (15 mg active/30 mgs Carbopol). The dosage utilized as shown in FIG. 4 was two capsules of 15 mg each. Other suitable excipients similar to Carbopol may also be utilized in oral formulations.

### Protocol

Protocol 1: The Effects of Pretreatment with Disaccharide Sodium and Hypersulfated Disaccharide on HNE induced reduction in TMV: After initial baseline TMV measurements were obtained, the animals were treated on separate occasions with disaccharide sodium (10 mg, 30 mg or 100 mg) or hypersulfated disaccharide (10 mg, 30 mg, or 100 mg). After 30 minutes, the sheep were then challenged with aerosolized HNE. Measurements of TMV were obtained 15 min, 30 min, and 45 min after HNE administration, and then hourly for up to 6 hours.

Protocol 2: The Effects of Hypersulfated Disaccharide on Reversing HNE induced reduction in TMV: After obtaining baseline TMV measurements, the sheep were challenged with aerosolized HNE. TMV measurements were then obtained hourly for the first four hours after administration of HNE. Immediately, after the 4 h TMV measurement, the sheep were treated with 10 mg, 30 mg or 100 mg of hypersulfated disaccharide. Serial TMV measurements were obtained hourly out to 8 h post HNE.

Protocol 3: The Effects of Oral Hypersulfated Disaccharide on HNE-induced Reduction in TMV: The animals were treated with two doses of oral hypersulfated disaccharide (14a) (2 capsules of 15 mg each with 30 mg Carbopol, with total dose of active equal to 30 mg), administered every 12 hours. The last dose was administered 90 minutes before aerosol challenge with HNE. Measurements of TMV were obtained for baseline and 15 minutes after challenge with aerolized HNE and then serially for up to six hours following challenge as described above.

### RESULTS

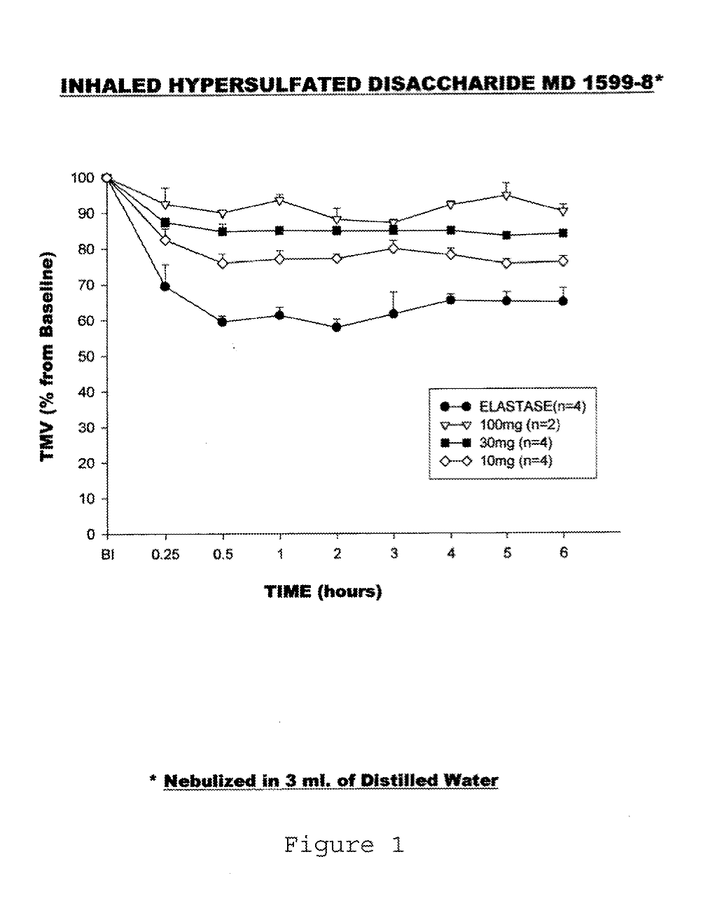

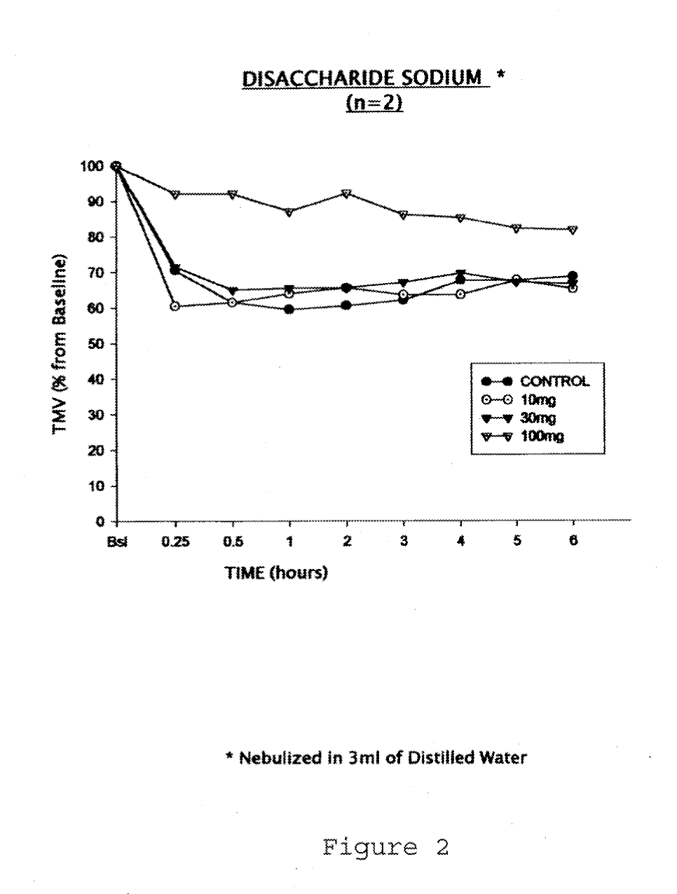

FIG. 1 illustrates the effects of inhaled hypersulfated disaccharide (compound 14a) on the HNE-induced reduction in TMV. HNE alone reduced TMV to ˜60% of baseline. Pretreatment with inhaled hypersulfated disaccharide resulted in a dose-dependent protection against this HNE induced reduction in TMV. In comparison FIG. 2 shows the effects of equivalent doses of 2′,6 disulfate disaccharide sodium (produced by chemical depolymerization of heparin with nitric oxide) on the HNE-induced effects. In contrast to hypersulfated disaccharide, neither the 10 mg nor 30 mg dose of the 2′,6-disulfate disaccharide sodium provided protection against the FINE-induced response. Thus, hypersulfated disaccharide (e.g. having more than 2 sulfates) showed increased potency in protecting against HNE-induced effects. The 2′,6-disulfate disaccharide sodium used in this comparative example is the identical compound shown as a compound of formula I with hydroxyl groups instead of the sulfate groups (i.e., R1 R2, R5 and R6 is H and R3 and R4 are sulfate and having the sodium salt of the carboxylate anion).

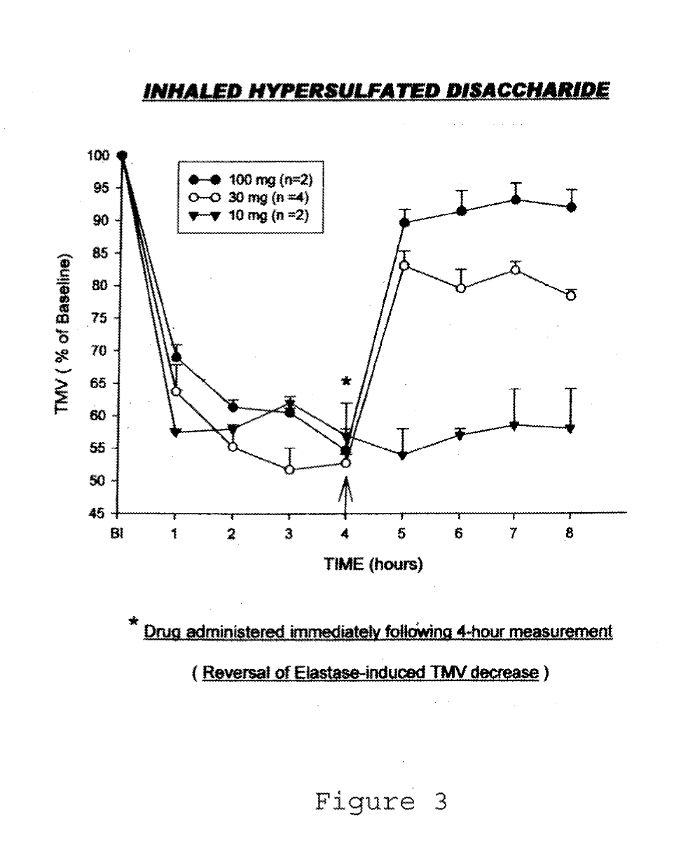

FIG. 3 illustrates that hypersulfated disaccharide can also reverse the effects of HNE. In this study, the 10 mg dose of hypersulfated disaccharide was ineffective, but significant reversal of the HNE-induced response was seen with both 30 mg and 100 mg of hypersulfated disaccharide. These findings indicate that hypersulfated disaccharide can be used therapeutically as well as prophylactically (as seen in FIG. 1) to combat HNE-induced reductions in TMV.

In the studies presented in FIGS. 1-3, the animal data clearly shows that the claimed compound is an effective modulator of diseases or conditions associated with human neutrophil elastase. In a preferred embodiment, the claimed compounds of formula I and salts thereof are in the form of a polysulfated salt and are delivered to the lungs of a patient in need of treatment thereof.

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize or be able to ascertain using no more than routine experimentation, numerous embodiments of the claimed invention which may not have been expressly described. Such embodiments are within the scope of the invention.

The present invention further relates to a method of treating an elastase related disorder with any polysulfated disaccharide including those disaccharides derived from heparin and which have the six-six ring structures and provided that at least three sulfate groups are present on the moiety. Such compounds are described in, for example, US patent publications US20030087875; U.S. Pat. Nos. 5,690,910; 6,193,957 and 7,056,898 all of which are incorporated by reference. The N-sulfated disaccharide unit shown below and polysulfated versions thereof including stereoisomers thereof are also effective in treating elastase related disorders:

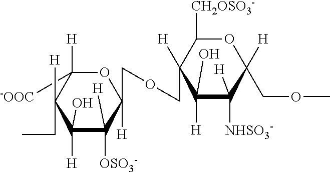

The term “hypersulfated disaccharide” thus means any disaccharide moiety having at least two sulfate moieties on the disaccharide core molecule and provided that such molecules do not include sodium disaccharide having R1, R2, R5 and R6 as Hand R3 and R4 as sulfate (SO₃⁻M⁺) in a compound of formula I. The term also includes any polysulfated disaccharide derived from heparin and having a low molecular weight (e.g. around 1,000 daltons or less) and any polysulfated derivative or chemically/enzymatically modified version thereof and provided that said moiety has at least two sulfate groups. Enzymatic treatment provides a 6,6 disaccharide as shown above. Chemical depolymerization with NO provides the 6,5 ring structure. Preferred modifications or derivatives have at least three sulfate moieties. The most preferred moieties have all hydroxyl groups replaced with sulfate groups and any N groups are N-sulfated.

### REFERENCES

Serisier et al. Inhaled heparin in Cystic Fibrosis Eur Respir J 2006; 27:354-358;

Birrell et al. Steroid-resistant Inflammation in a Rat Model of Chronic Obstructive Pulmonary Disease is Associated with a Lack of Nuclear Factor-κB Pathway Activation Am J Respir Crit Care Med vol 172. Pp 74-84, 2005;

Michele et al. The Safety of Tiopropium-The FDA's Conculsions N. Engl. J Med 363;12 Sep. 16, 2010;

Jancin New Therapies Begin to Fill CF Pipeline

Sabater et al. Comparative Effects of Salmeterol, Albuterol, and Ipratropium on Normal and Impaired Mucociliary Function in Sheep Chest 2005; 128; 3743-3749;

Spencer et al. New Insights Into the Inhibition of Human Neutrophil Elastase by Heparin Biochemistry 2006, 45, 9104-9120;

Volpi Inhibition of Human Leukocyte Elastase Activity by Heparins: Influence of Charge Density Biochimica et Biophysica Acta 1290 (1996) 299-307;

Sissi et al., Interactions of Low-Molecular Weight Semi-Synthetic Sulfated Heparins with Human Leukocyte Elastase and Human Cathepsin G Biochemical Pharmacology 71 (2006) 287-293;

Lever et al., Size-Fractionated Heparins Have Differential Effects on Human Neutrophil Function in Vitro British Journal of Pharmacology (2007) 151, 837-843;

Redini et al. Inhibition of Leucocyte Elastase by Heparin and Its Derivatives Biochem. J. (1988), 252, 515-51.9;

Ledson et al., Nebulized Heparin in Burkholderia cepacia Colonized Adult Cystic Fibrosis Patients Eur Respir J 2001; 17: 36-38;

Lafuma et al., Prevention of Leucocyte Elastase-Induced Emphysema in Mice by Heparin Fragments Eur Respir J 1991, 4, 1004-1009;

Birrell et al., Steroid-Resitant Inflammation in a Rat Model of chronic Obstructie Pulmonary Disease is Associated with a Lack of Nuclear Factor-KappaB Pathway Activation Am J Respir Crit Care Med, 2005 Jul. 1; 172(1):74-84. Epub 2005 Apr. 1.

Spencer et al. New Insights Into the Inhibition of Human Neutrophil Elastase by Heparin Biochemistry, 2006 Aug. 1; 45(30):9104-20;

Lee et al. Leukocyte Elastase Physiological Functions and Role in Acute Lung Injury Am J Respir Crit Care Med vol 164, pp 896-904 (2001);

Walsh et al. Heparin and Heparan Sulphate are Inhibitors of Human Leukocyte Elastase Clinical Science (1991) 81, 341-346;

Redini et al. Influence of Heparin Fragments on the Biological Activities of Elastase(s) and α₁ Proteinase Inhibitor Biochemical Pharmacology Vol. 37, No. 22, pp. 4257-4261 1988;

Lonky et al., Stimulation of Human Granulocyte Elastase by Platelet Factor 4 and Heparin Biochemical and Biophysical Research Communications Vol. 85, No. 3, 1978;

Cadene et al., Influence of Low Molecular Mass Heparin on the Kinetics of Neutrophil Elastase Inhibition by Mucus Proteinase Inhibitor The Journal of Biological Chemistry 270, No. 22 June 2, 13204-13209, 1995;

Finotti et al., Differential Effects of Heparin and Glucose on Structural Conformation of Human α1 Antitrypsin: Evidence for a Heparin-Induced Cleaved Form of the Inhibitor Archives of Biochemistry and Biophysics 347, 1, Nov. 1, 19-29, 1997;

Faller et al., Heparin Interferes with the Inhibition of Neutrophil Elastase by its Physiological Inhibitors Biol. Chem. 373, 503-508, July 1992;

Siedle et al., Natural Compounds as Inhibitors of Human Neutrophil Elastase Planta Med. 2007 May; 73(5):401-20;

Kelly et al., Targeting Nuetrophil Elastase in Cystic Fibrosis Expert Opin Ther Targets, 2008 Feb; 12(2):145-57;

Ohbayashi H. Neutrophil Elastase Inhibitors as Treatment for COPD 2002 Jul; 11(7):965-80;

Kawabala et al., Pharmacological Profile of a Specific Neutrophil Elastase Inhibitor, Sivelestat Sodium Hydrate 2003 Aug; 122(2):151-60;

Hagiwara et al. A Neutrophil Elastase Inhibitor, Sivelestat, Reduces Lung Injury Following Endotoxin Induced Shock in Rats by Inhibiting HMGB1 Inflammation, 2008 Aug.; 31(4):227-34;

Bessho et al. Sivelestat Reduces Myocardial Ischemia and Reperfusion Injury in Rate Hearts Even When Administered After Onset of Myocardial Ischemia Interact Cardiovasc Thorac Surg 2009 Jun; 8(6):629-34, Epub 2009 Mar. 11;

Roghanian et al., Neutrophil Elastase (NE) and NE Inhibitors: Canonical and Noncanonical Functions in Lung Chronic Inflammatory Diseases (Cystic Fibrosis and Chronic Obstructive Pulmonary Disease) J Aerosol Med Pulm Drug Deliv. 2008 Mar; 21(1):125-44;

Hagiwara et al., Neutrophil Elastase Inhibitor (sivelestat) Reduces the Levels of Inflammatory Mediators by Inhibiting NF-κB Inflamm Res. 2009 Apr; 58(4): 198-203;

Becker et al. Inhibition of PMN-Elastase Activity by Semisynthetic Glucan Sulfates 2003 May; 89(5):915-25;

1. A pharmaceutical formulation suitable for delivery to the lungs of a patient in need of such treatment comprising a compound of formula I and pharmaceutically acceptable salts thereof 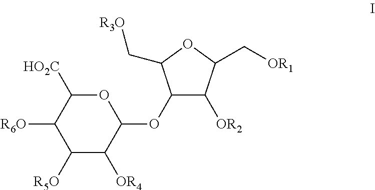
wherein R₁, R₂, R₃, R₄, R₅ and R₆ are independently selected from the group consisting of H, SO₃H or PO₃H and provided that at least two of R₁-R₆ is selected from SO₃H or PO₃H.

2. The formulation according to claim 1 wherein at least three of R₁-R₆ is selected from SO₃H or PO₃H.

3. The formulation according to claim 1 wherein at least four of R₁-R₆ is selected from SO₃H or PO₃H.

4. The formulation according to claim 1 wherein at least five of R₁-R₆ is selected from SO₃H or PO₃H.

5. The formulation according to claim 1 wherein R₁-R₆ is selected from SO₃H or PO₃H.

6. A dry-powder pharmaceutical formulation comprising
(i) a compound of formula I and pharmaceutically acceptable salts thereof 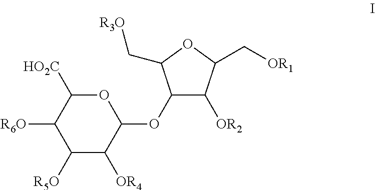
wherein R₁, R₂, R₃, R₄, R₅ and R₆ are independently selected from the group consisting of H, SO₃H or PO₃H and provided that at least two of R₁-R₆ is selected from SO₃H or PO₃H.

7. A nebulizable pharmaceutical formulation comprising (i) a compound of formula II 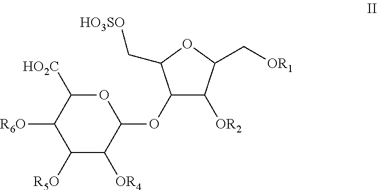
and pharmaceutically acceptable salts thereof wherein R₁, R_{2,} R₅ and R₆ are independently selected from the group consisting of H, SO₃H or PO₃H; R₄ is selected from SO₃H or PO₃H and
(ii) a delivery agent.

8. An aerosol, dry powder or nebulizable formulation according to claim 1 wherein the compound of formula I is selected from a compound having R₁-R₆ as

| | |
|---|---|
| | I |
| 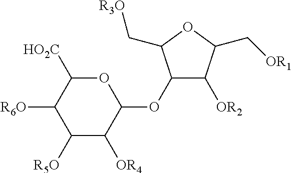 | |
| R₁ | R₂ | R₃ | R₄ | R₅ | R₆ |
|---|---|---|---|---|---|
| —SO₃H | H | —SO₃H | —SO₃H | H | H |
| H | —SO₃H | —SO₃H | —SO₃H | H | H |
| H | H | —SO₃H | —SO₃H | H | —SO₃H |
| —SO₃H | H | —SO₃H | —SO₃H | —SO₃H | H |
| —SO₃H | H | —SO₃H | —SO₃H | H | —SO₃H |
| H | H | —SO₃H | —SO₃H | —SO₃H | —SO₃H |
| —SO₃H | H | —SO₃H | —SO₃H | —SO₃H | —SO₃H |
| —SO₃H | —SO₃H | —SO₃H | —SO₃H | H | H |
| H | —SO₃H | —SO₃H | —SO₃H | —SO₃H | H |
| H | —SO₃H | —SO₃H | —SO₃H | H | —SO₃H |
| —SO₃H | —SO₃H | —SO₃H | —SO₃H | —SO₃H | H |
| —SO₃H | —SO₃H | —SO₃H | —SO₃H | H | —SO₃H |
| H | —SO₃H | —SO₃H | —SO₃H | —SO₃H | —SO₃H |
| —SO₃H | —SO₃H | —SO₃H | —SO₃H | —SO₃H | —SO₃H |

and pharmaceutically acceptable salts thereof.

9. A method of treating or alleviating an elastase related condition in an animal in need of treatment thereof comprising administration of
(i) a pharmaceutically effective amount of a formulation comprising a compound of formula I 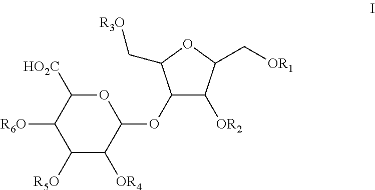
and pharmaceutically acceptable salts thereof wherein R₁-R₆ are independently selected from SO₃H, PO₃H or H and provided that at least two of R₁-R₆ is SO₃H or PO₃H.

10. The method according to claim 9 wherein the formulation is an aerosol formulation.

11. The method according to claim 9 wherein at least three of R₁-R₆ is selected from SO₃H or PO₃H.

12. The method according to claim 9 wherein at least four of R₁-R₆ is selected from SO3H or PO₃H.

13. The method according to claim 9 wherein at least five of R₁-R₆ is selected from SO₃H or PO₃H.

14. The method according to claim 9 wherein R₁-R₆ is selected from SO₃H.

15. The method according to claim 9 wherein the elastase-related condition is bronchitis.

16. The method according to claim 9 wherein the elastase-related condition is emphysema.

17. The method according to claim 9 wherein the elastase-related condition is COPD.

18. The method according to claim 9 wherein the elastase-related condition is cystic fibrosis.

19. The method according to claim 9 wherein the elastase-related condition is acute respiratory distress syndrome and the formulation is delivered via intravenous administration.

20. A delivery device for pulmonary delivery of:
a compound of formula I or a pharmaceutically acceptable salt thereof 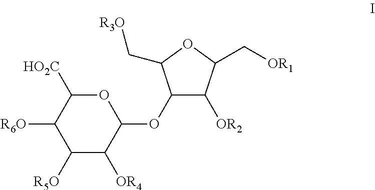
wherein R₁-R₆ are independently selected from SO₃H, PO₃H or H and provided that at least two of R₁-R₆ is SO₃H or PO₃H.

21. A combination for the treatment of an elastase associated disorder comprising a compound of formula I according to claim 1 and at least one additional active ingredient selected from anti-inflammatory agents, leukotriene antagonists or modifiers, anticholinergic drugs, mast cell stabilizers, corticosteroids, immunomodulators, beta-adrenergic agonists (short acting and long acting), methyl xanthines, leukotriene antagonists and antihistamines.

22. The combination according to claim 21 wherein the at least one additional active ingredient is selected from montelukast sodium, albuterol; levoalbuterol; salmeterol; formoterol, fluticasone propionate; budesonide; ceterizine; loratadine; desloratadine; theophylline, ipratropium, cromolyn, nedocromil, beclomethasone, flunisolide, mometasone, triaminoclone, prednisoline, prednisone, zafirlukast, zileuton or omalziunab.

23. A method of treating an elastase-related disorder with a hypersulfated disaccharide.

24. The method according to claim 23 wherein the core ring is a 6,6 disaccharide or a 6,5 disaccharide.

25. The method according to claim 23 wherein the disaccharide is a 6,5 disaccharide moiety.

26. A method of treating an elastase-related disorder comprising administering an oral formulation of a hypersulfated disaccharide.
